(19) United States
(12) Patent Application Publication
(10) Pub. No.: US 2016/0114111 A1
(43) Pub. Date: Apr. 28, 2016

(54) Nebulizer mouthpiece

(71) Applicant: HSINER Co., Ltd., Taichung City (TW)

(72) Inventor: Eric Chang, Taichung City (TW)

(21) Appl. No.: 14/757,461

(22) Filed: Dec. 23, 2015

Related U.S. Application Data
(63) Continuation-in-part of application No. 14/025,212, filed on Sep. 12, 2013.

(51) Int. Cl. A61M 11/00; A61M 15/00

(52) U.S. Cl. CPC A61M 11/006; A61M 15/0021

(57) ABSTRACT

A nebulizer mouthpiece includes a hollow main body and a connecting tube. The main body is a one-piece element, and includes an annular wall defining an inner space and having a middle section, an outlet section, and a storage section connected respectively to two ends of the middle section. The inner space has a storage space part that is located between the middle section and the storage section, and that has a volume ranging between 60 and 100 milliliters. The connecting tube is adapted for interconnecting the middle section of said main body and a liquid container, and surrounding a central axis. A distance between the central axis and a distal end of the storage section is not greater than 100 millimeters.

### CROSS-REFERENCE TO RELATED APPLICATION

This application is a continuation-in-part (CIP) of co-pending U.S. patent application Ser. No. 14/025,212, filed on Sep. 12, 2013, and abandoned as of the filing date of this application.

### FIELD

The disclosure relates to medical equipment, more particularly to a nebulizer mouthpiece.

### BACKGROUND

Referring to FIG. 1, a conventional nebulizer mouthpiece 1 is used with a container 2 and a pipe 3. Liquid-form medicine is stored inside the container 2, and is transported from the container 2 through the conventional nebulizer mouthpiece 1 and into a user's mouth as mist-form by pressured gas released through the pipe 3.

Referring to FIG. 2, the conventional nebulizer mouthpiece 1 includes a T-shaped connecting tube 101, a mouth-receiving piece 102, and a bellows tube 103. The container 2, the mouth-receiving piece 102 and the bellows tube 103 are respectively connected to the three tube sections 105, 106, 104 of the connecting tube 101. The connecting tube 101 and the bellows tube 103 are standard products, and the inner diameter of the section 104 for connecting the bellows tube 103 is 20 millimeters.

Since the bellows tube 103 is corrugated, since the smallest inner diameter of the bellows tube 103 is smaller than that of the sections 104, 106, and since the mouth-receiving piece 102 is tapered away from the section 106, once the connecting tube 101, the mouth-receiving portion 102 and the bellows tube 103 are connected together, the inner annular surface of the conventional nebulizer mouthpiece 1 in a horizontal direction initially goes through a repeated increasing/decreasing change in the bellows tube 103, then increases from the bellows tube 103 to the section 104 and then decreases in the mouth-receiving piece 102 thus leading to the following drawbacks.

a) During inhalation and exhalation, one part of the mist-form medicine is transported to the conventional nebulizer mouthpiece 1 flowing into the connecting tube 101 for the user to inhale, while the other part of the mist-form medicine remains inside the section 104 of the connecting tube 101 and the bellows tube 103. Generally, the amount of an inhalation of a person is about 60 milliliters, so that when a length of the section 104 of the connecting tube 101 and a length of the bellows tube 103 are too long, the distance that the other part of the mist-form medicine has to travel to the connecting tube 101 will be too long, and the total amount of the mist-form medicine inhaled by the user will be insufficient and the effectiveness of a dose will be affected.

b) The inner diameter of the section 104 is 20 millimeters, so that when the medicine is transported from the container 2 through the section 105 into the conventional nebulizer mouthpiece 1 under high pressure, the mist-form medicine will come in contact with the top side of the section 104 and may even condense into liquid form and flow back into the container 2, thereby reducing the amount of mist-form medicine inhaled by the user and affecting its dose and effect.

c) Due to the inconsistent inner diameter of the conventional nebulizer mouthpiece 1 in the horizontal direction (i.e., from small to large to small) not only might the mist-form medicine be trapped and turned into liquid form, but unbalanced and disturbing air flow may also be created.

d) The length of the bellows tube 103 is too long and easily interferes with nearby objects and people, causing discomfort and inconvenience.

### SUMMARY

Therefore, the object of the disclosure is to provide a nebular mouthpiece that can eliminate at least one of the aforesaid drawbacks of the prior art.

Accordingly, there is provided a nebulizer mouthpiece including a hollow main body and a connecting tube. The main body is formed into a one-piece element, and includes an annular wall. The annular wall defines an inner space and has a middle section, an outlet section, and a storage section. The outlet section is connected to an end of the middle section. The storage section is connected to another end of the middle section. The inner space has a storage space part that is located between the middle section and the storage section, and that has a volume ranging between 60 and 100 milliliters .

The connecting tube is adapted for interconnecting the middle section of the main body and a liquid container, and surrounds a central axis. A distance between the central axis and a distal end of the storage section is not greater than 100 millimeters. Liquid received in the liquid container is turned into mist and introduced into the inner space through the connecting tube.

### BRIEF DESCRIPTION OF THE DRAWINGS

Other features and advantages of the disclosure will become apparent in the following detailed description of the preferred embodiment with reference to the accompanying drawings, of which:

FIG. 1 is a schematic view of a conventional nebulizer mouthpiece adapted to be used with a container and a pipe;

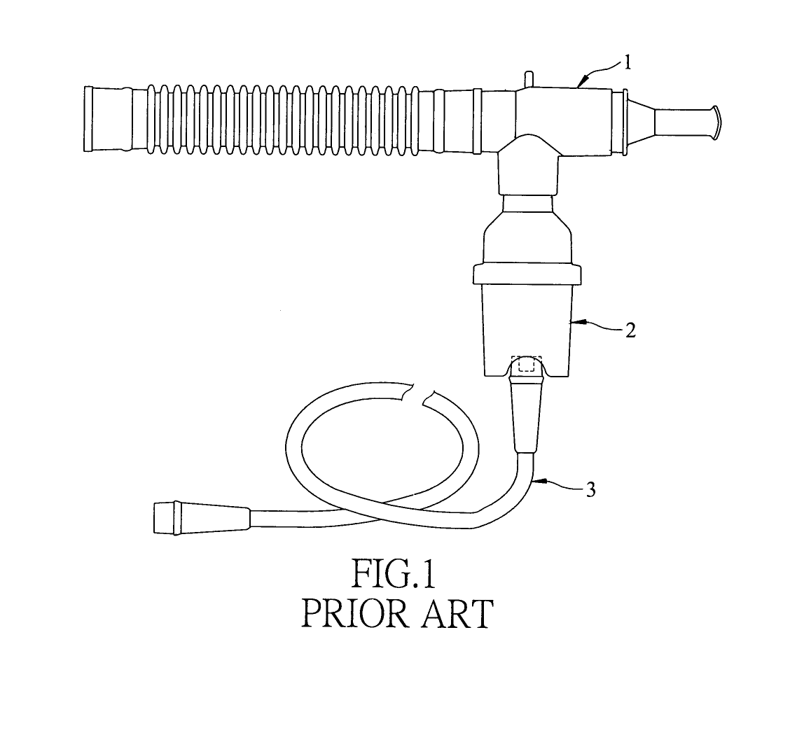

FIG. 2 is an exploded schematic view of the conventional nebulizer mouthpiece;

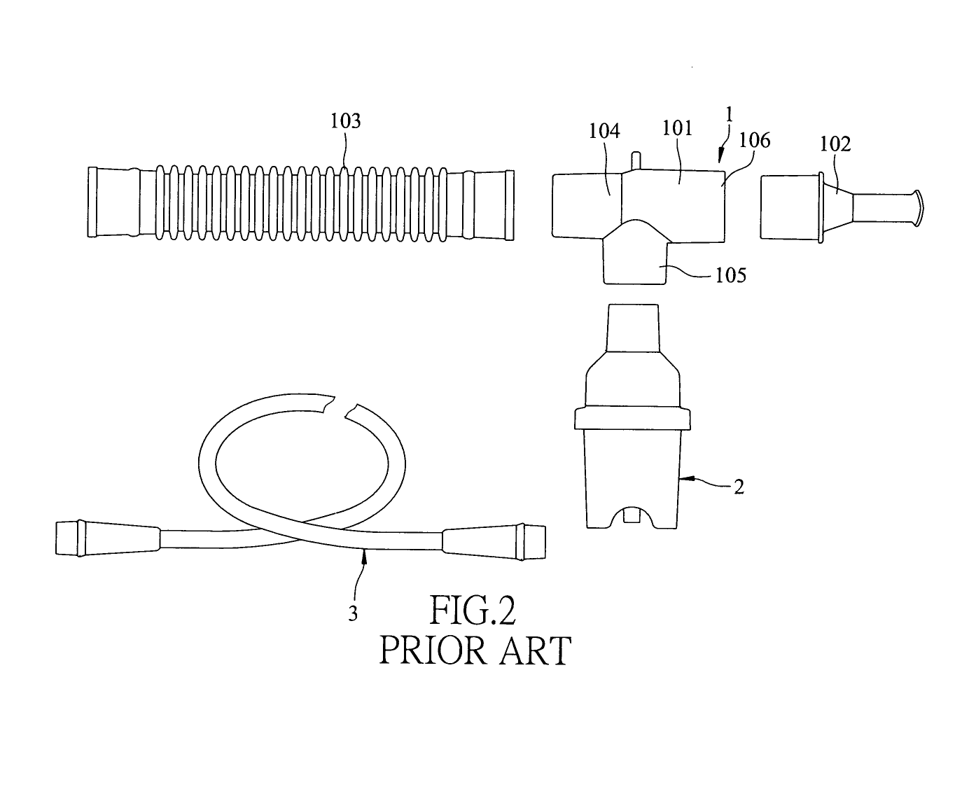

FIG. 3 is an exploded view of an assembly of a preferred embodiment of a nebulizer mouthpiece according to the disclosure, a liquid container, and a pipe; and

FIG. 4 is a schematic view of the assembly.

### DETAILED DESCRIPTION

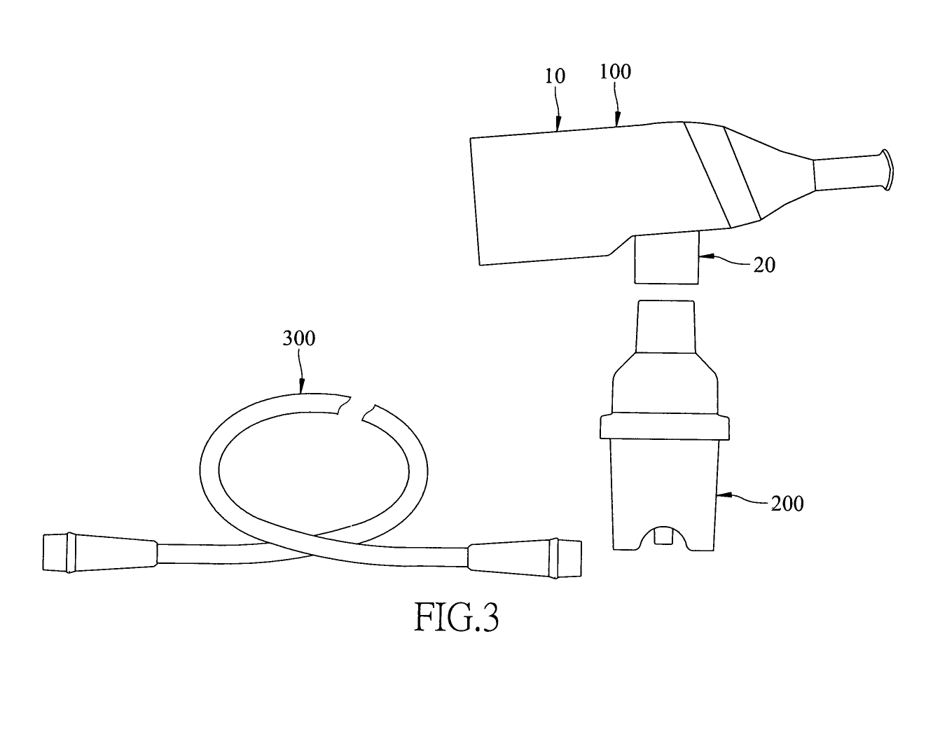

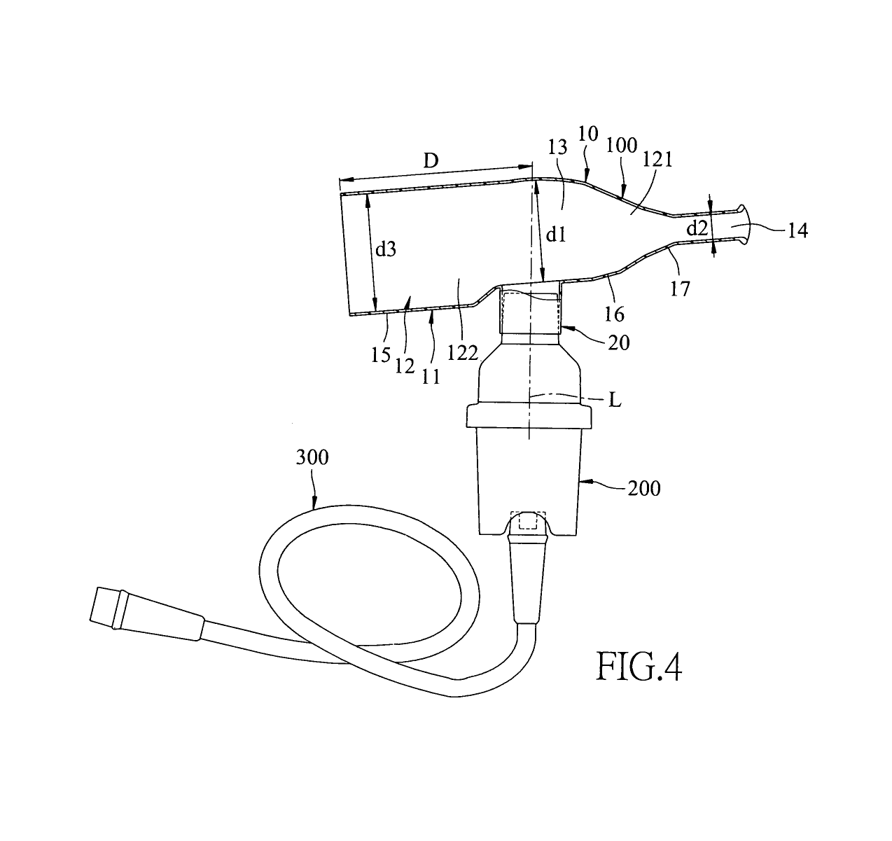

Referring to FIGS. 3 and 4, a preferred embodiment of a nebulizer mouthpiece 100 according to the disclosure is adapted to be used with a liquid container 200 and a pipe 300. The nebulizer mouthpiece 100 includes a hollow main body 10 and a connecting tube 20.

The main body 10 is formed into a one-piece element, and includes an annular wall 11. The annular wall 11 defines an inner space 12, and has a middle section 13, an outlet section 14, a storage section 15, a connecting section 16 and a guide section 17. The outlet section 14 is connected to an end of the middle section 13 by the connecting section 16 which is curved in a longitudinal direction of the main body 10, and the guide section 17 which is generally frustoconical and tapers from the middle section 13 toward the outlet section 14. The storage section 15 is connected to another end of the middle section 13. The inner space 12 has an outlet space part 121 that is located between the middle section 13 and the outlet section 14, and a storage space part 122 that is located between the middle section 13 and the storage section 15. The storage space part 122 has a volume ranging between 60 and 100 milliliters. The middle section 13 is aligned with the connecting tube 20, and has a smooth inner surface and an inner diameter (d1). The outlet section 14 has an internal diameter (d2) that is smaller than the inner diameter (d1) of the middle section 13. The storage section 15 has a smooth inner surface facing the inner space 12 and an interior diameter (d3) greater than the inner diameter (d1) of the middle section 13.

The connecting tube 20 is adapted for interconnecting the middle section 13 of the main body 10 and the liquid container 200 and surrounds a central axis (L). A distance (D) between the central axis (L) and a distal end of the storage section 15 is not greater than three times the inner diameter (d1) of the middle section 13. Liquid received in the liquid container 200 is turned into mist and introduced into the inner space 12 through the connecting tube 20. In this embodiment, the inner diameter (d1) of the middle section 13 is not smaller than 30 millimeters, and the distance (D) is not greater than 100 millimeters. The main body 10 and the connecting tube 20 are integrally formed into one piece.

When the liquid container 200 contains liquid-form medicine, the pipe 300 uses pressurized gas to turn the liquid-form medicine into mist-form, which is then transported through the connecting tube 20 into the nebulizer mouthpiece 100. One part of the mist-form medicine flows through the middle section 13, the connecting and guide sections 16, 17, and out into a user's mouth through the outlet section 14; the other part of the mist-form medicine flows to the storage section 15 and remains inside the storage space part 122. The volume of the storage space part 122 ranges between 60 and 100 milliliters, and the distance (D) is not greater than 100 millimeters. Since the amount of an inhalation of an average person is about 60 milliliters, the other part of the mist-form medicine which flows to the storage section 15 can also be effectively inhaled, thereby increasing the total amount of the mist-form medicine inhaled by the user and enhancing the effectiveness of the dose.

Additional advantages of one disclosure can be summarized as follows.
a) Since the inner diameter (d1) of the middle section 13 is not smaller than 30 millimeters, when the mist-form medicine is guided into the middle section 13, direct contact against the middle section 13 is reduced due to the greater distance available, preventing the mist form medicine from returning to its liquid form and ensuring that the user will inhale the effective amount of the medicine.
b) Since the inner surfaces of the middle section 13 and the storage section 15 are smooth, and since the transition of inner diameter of the connecting section 16 and the guide section 17 toward the outlet section 14 is also smooth, the flow of the mist-form medicine runs smoother (i.e., without being blocked by or caught between any dead angles created by abrupt depressions or protrusions) into the user's mouth.
c) A length of the storage section 15 is short so as to prevent the nebulizer mouthpiece 100 from interfering with nearby objects and minimizing discomfort.

It is worth mentioning that even though the main body 10 is a one-piece structure that is simple and easy to manufacture, it allows the mist-form medicine to be guided even more smoothly than the prior art, which enhances the effectiveness of the dose inhaled.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A nebulizer mouthpiece comprising:
a hollow main body formed into a one-piece element, and including an annular wall that defines an inner space and that has a middle section, an outlet section connected to an end of said middle section, and a storage section connected to another end of said middle section, said inner space having a storage space part that is located between said middle section and said storage section, and that has a volume ranging between 60 and 100 milliliters; and
a connecting tube adapted for interconnecting said middle section of said main body and a liquid container and surrounding a central axis, a distance between the central axis and a distal end of said storage section being not greater than 100 millimeters;
wherein liquid received in the liquid container is turned into mist and introduced into said inner space through said connecting tube.

2. The nebulizer mouthpiece as claimed in claim 1, wherein said middle section has an inner diameter, said outlet section of said main body having an internal diameter that is smaller than the inner diameter, said storage section having an interior diameter greater than the inner diameter.

3. The nebulizer mouthpiece as claimed in claim 2, wherein said annular wall of said main body further has a guide section interconnecting said middle section and said outlet section, and tapering toward said outlet section.

4. The nebulizer mouthpiece as claimed in claim 1, wherein said main body and said connecting tube are integrally formed into one piece.

5. The nebulizer mouthpiece as claimed in claim 1, wherein said storage section of said main body has a smooth inner surface facing said inner space.
